(19) United States
(12) Patent Application Publication
(10) Pub. No.: US 2024/0374383 A1
(43) Pub. Date: Nov. 14, 2024

(54) REPLACEMENT HEART VALVE

(71) Applicant: Edwards Lifesciences CardiAQ LLC, Irvine, CA (US)

(72) Inventors: Jeremy Brent Ratz, Winchester, MA (US); Luca Pesce, Huntington Beach, CA (US); Glen T. Rabito, Lake Forest, CA (US); Christine Thanh Nguyen, Santa Ana, CA (US)

(21) Appl. No.: 18/779,022

(22) Filed: Jul. 21, 2024

Related U.S. Application Data
(63) Continuation of application No. 17/357,816, filed on Jun. 24, 2021, now Pat. No. 12,083,011.
Application No. 17/357,816 is a continuation of application No. 16/247,403, filed on Jan. 14, 2019, now Pat. No. 11,045,313.
Application No. 16/247,403 is a continuation of application No. 15/462,438, filed on Mar. 17, 2017, now Pat. No. 10,179,044.
Application No. 15/462,438 is a continuation of application No. 14/716,507, filed on May 19, 2015, now abandoned.
(60) Provisional application No. 62/000,309, filed on May 19, 2014.

(51) Int. Cl. A61F 2/24

(52) U.S. Cl. CPC A61F 2/2418; A61F 2/2445; A61F 2220/0008; A61F 2220/005; A61F 2220/0075; A61F 2250/0039; A61F 2250/0069

(57) ABSTRACT

A prosthesis for placement within a native heart valve preferably includes an expandable frame having locking tabs disposed along a proximal end. A plurality of distal anchors is disposed along a distal end of the frame. Each distal anchor is provided with a cushion cover. An outer fabric member surrounds the frame and may extend outwardly from the frame in an expanded condition for providing an improved seal in the region of a native annulus. A valve body is positioned within an interior of the expandable from for allowing blood flow in only one direction. In preferred embodiments, the cushions are positioned over enlarged head portions of the distal anchors for further reducing trauma on surrounding tissue.

### CROSS-REFERENCE TO RELATED APPLICATIONS

This application is a continuation of U.S. application Ser. No. 17/357,816, filed Jun. 24, 2021, which is a continuation of U.S. application Ser. No. 16/247,403, filed Jan. 14, 2019, now U.S. Pat. No. 11,045,313, which is a continuation of U.S. application Ser. No. 15/462,438, filed Mar. 17, 2017, now U.S. Pat. No. 10,179,044, which is a continuation of U.S. application Ser. No. 14/716,507, filed May 19, 2015, now abandoned, which claims priority to U.S. Provisional Application No. 62/000,309 filed May 19, 2014, which is hereby incorporated herein by reference in its entirety and is to be considered a part of this specification.

### BACKGROUND

### Field

Certain embodiments disclosed herein relate generally to prostheses for implantation within a lumen or body cavity. In particular, certain embodiments relate to expandable prostheses such as replacement heart valves, such as for the mitral valve, that are configured to atraumatically grasp intralumenal tissue.

### Background

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves are often intended to at least partially block blood flow. However, a problem occurs when blood flows around the valve on the outside of the prosthesis. For example, in the context of replacement heart valves, paravalvular leakage has proven particularly challenging. An additional challenge relates to the ability of such prostheses to be secured relative to intralumenal tissue, e.g., tissue within any body lumen or cavity, in an atraumatic manner. Further challenges arise when trying to controllably deliver and secure such prostheses in a location such as at a native mitral valve.

### SUMMARY OF THE INVENTION

Embodiments of the present disclosure are directed to a prosthesis, such as but not limited to a replacement heart valve. According to some embodiments, a prosthesis can be configured to be deployed within a body cavity and prevent axial flow of fluid around an exterior of the prosthesis. The prosthesis can include an expandable frame configured to radially expand and contract for deployment within the body cavity, and an annular flap positioned around an exterior of the expandable frame. Further embodiments are directed to methods of delivering a prosthesis, e.g. a replacement heart valve, and methods of using a prosthesis to create a barrier to fluid flow exterior to the prosthesis (e.g., to prevent paravalvular leakage).

In some embodiments, the prosthesis can include an expandable frame having a proximal end and a distal end and a longitudinal axis extending therethrough. In some embodiments, the frame can be designed to radially expand and contract for deployment within the body cavity. The prosthesis can include an annular flap positioned around and secured to an exterior of the frame. The annular flap may have a distal edge secured at or near the distal end of the frame and extending to a proximal edge secured at an intermediate location on the frame between the proximal and distal ends. The prosthesis can include a valve body positioned within an interior of the expandable frame. In some embodiments, the valve body can include an inner skirt secured to the interior of the expandable frame and a plurality of leaflets designed to allow flow in a first direction and prevent flow in a second opposite direction. In some embodiments, an opening is defined at or near the distal end of the frame between the annular flap and the valve body which can provide access for fluid to flow into a space between the annular flap and the valve body. In some embodiments, the fluid flow into the space can cause the annular flap to move from a first configuration wherein the flap is closer to the frame to a second configuration wherein the flap is spaced further away from the frame to increase the surface area of the prosthesis and create a barrier to fluid flow exterior to the frame when deployed within the body cavity.

### BRIEF DESCRIPTION OF THE DRAWINGS

FIG. 1A is a proximal oriented, perspective view of an embodiment of a prosthesis illustrating a frame, a plurality of anchors, a band, a flap, and a valve body.

FIG. 1B is a distal oriented, perspective view of the prosthesis of FIG. 1A.

FIG. 2 is a front elevation view of the prosthesis of FIG. 1.

FIG. 3 is a front elevation view of another embodiment of a prosthesis.

FIG. 4 is a front elevation view of an embodiment of a frame.

FIG. 5 is a perspective view of an embodiment of an annular flap.

FIG. 6 is a front elevation view of the annular flap of FIG. 5.

FIG. 7 is a perspective view of an embodiment of a valve body.

FIG. 8 is a front perspective view of the valve body of FIG. 7.

FIG. 9 is a front elevation of an embodiment of a prosthesis illustrating a frame, a plurality of anchors, a band, a flap, and a valve body.

FIG. 10 is a front elevation another embodiment of a prosthesis.

FIG. 11A is a partial cross-sectional view of the prosthesis of FIG. 1 with the annular flap in a first configuration.

FIG. 11B is a partial cross-sectional view of the prosthesis of FIG. 11A with the annular flap in a first configuration.

FIG. 12A is a partial cross-sectional view of the prosthesis of FIG. 1 with the annular flap in a first configuration, the valve body being removed.

FIG. 12B is a partial cross-sectional view of the prosthesis of FIG. 12A with the annular flap in a first configuration.

FIG. 13A-15 illustrate schematic representations of the prosthesis of FIG. 3 positioned within a heart, with FIGS. 13A-13C illustrating the prosthesis in situ with distal anchors contacting the ventricular side of a mitral valve annulus, FIGS. 14A-14B illustrating the prosthesis in situ with distal anchors not contacting the ventricular side of the mitral valve annulus, and FIG. 15 illustrating the prosthesis in situ with distal anchors not extending between the chordae tendineae.

### DETAILED DESCRIPTION OF THE PREFERRED EMBODIMENTS

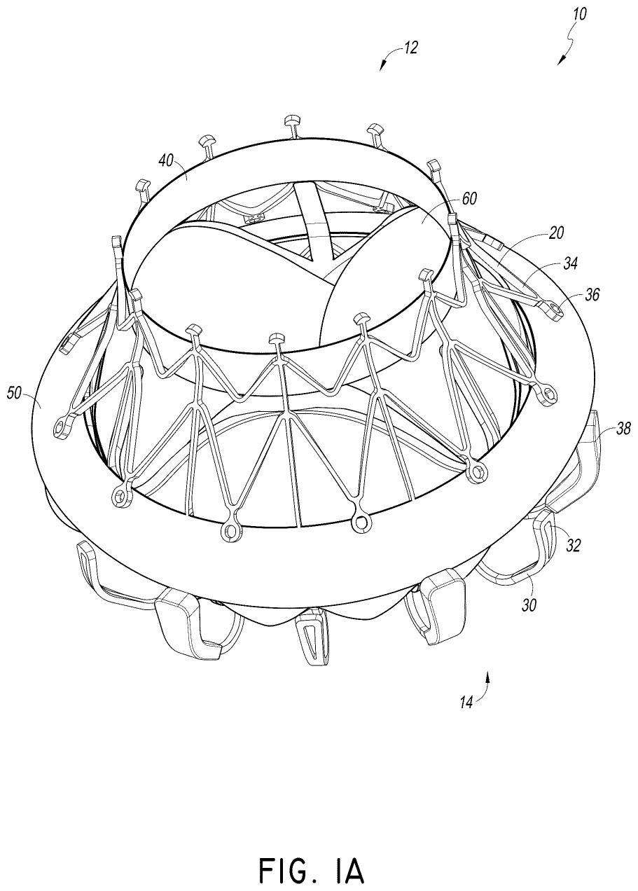

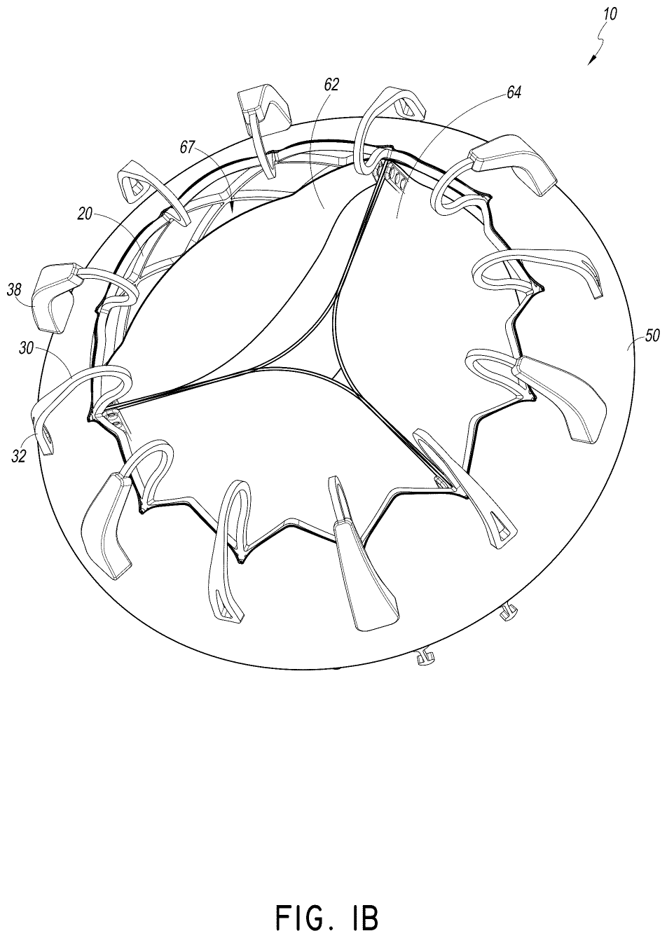

The embodiment of FIGS. 1A-4 illustrates a prosthesis 10. The prosthesis 10 can have components, features, and/or functionality similar to those described in any of U.S. Publication Nos. 2014/0277390, 2014/0277422, and 2014/0277427, the entire contents of all which are incorporated by reference herein. With reference first to the embodiments of FIGS. 1A-4, the prosthesis 10 can include a frame 20, anchors 30, 34, a band 40, an annular flap or sail 50 and a valve body 60. The prosthesis 10 can include a proximal end 12 and a distal end 14 with openings defined at both ends 12, 14 such that fluid can flow therethrough. In some embodiments, the proximal end 12 can be placed in the left atrium while the distal end 14 can be placed in the left ventricle such that prosthesis 10 can function as a replacement for a mitral valve. As will be discussed in greater detail below and as discussed in U.S. Publication Nos. 2014/0277390, 2014/0277422, and 2014/0277427, the prosthesis 10 can allow blood flow in a first direction from the proximal end 12 to the distal end 14 while preventing blood flow in a second direction from the distal end 14 to the proximal end 12. For example, during diastole the valve body 60 may be open to allow blood flow from the proximal end 12 to the distal end 14, and during systole the valve body 60 may be closed to prevent blood flow from the distal end 14 to the proximal end 12.

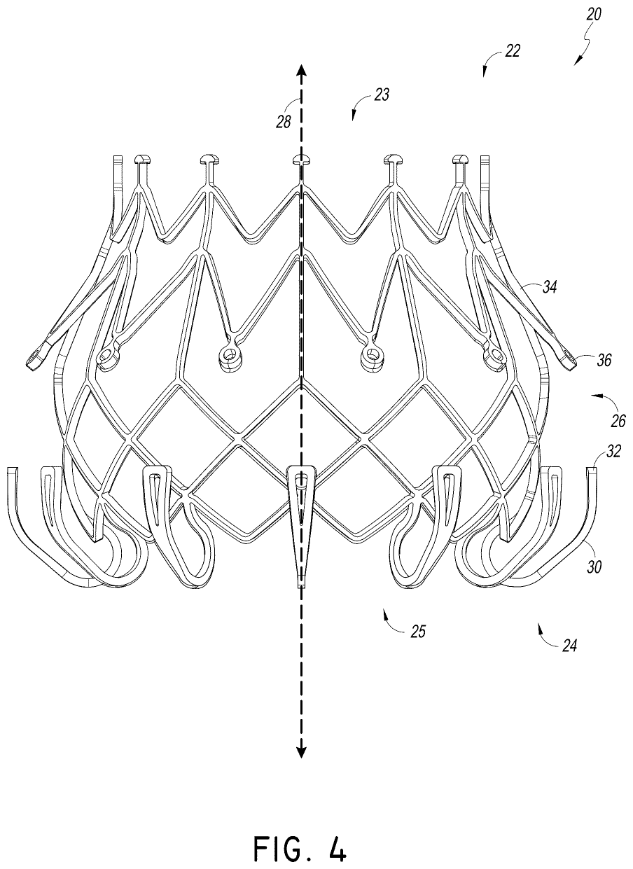

With reference now to the embodiment of FIG. 4, the embodiment illustrates an expandable frame 20 of the prosthesis 10 which can have a proximal end 22 and a distal end 24. In some embodiments, such as the illustrated embodiment, the frame 20 can include an intermediate portion 26 which has a greater diameter than the diameter of the frame 20 at the proximal and/or distal ends 22, 24 when the frame 20 is in an expanded configuration. In some embodiments, such as the illustrated embodiment, the frame 20 can include an intermediate portion 26 which has a greater cross-sectional area than the cross-sectional area of the frame 20 at the proximal and/or distal ends 22, 24 when the frame 20 is in an expanded configuration. The frame 20 can be designed to expand radially and contract for deployment within a body cavity, such as at a heart valve location such as the mitral valve. For example, as described in greater detail in U.S. Publication Nos. 2014/0277390, 2014/0277422, and 2014/0277427, the frame 20 can include a plurality of struts which define a plurality of foreshortening cells. In some embodiments, the frame 20 can be designed to radially and contract radially from a longitudinal axis 28 extending through the frame 20. As illustrated in the embodiments of FIGS. 1-4, the proximal end 22 can define a proximal opening 23 and the distal end 24 can define a distal opening 25.

With continued reference to the embodiments of FIGS. 1A-4 which illustrates the prosthesis 10, in some embodiments the prosthesis 10 can include one or more distal anchors 30. The distal anchors 30 can be positioned along or proximate a distal end 24 of the frame 20 and can be connected to the frame 20. The distal anchors 30 can be designed such that when the frame 20 is in an expanded configuration an end or tip 32 of each distal anchor 30 is positioned radially outward from the frame 20 and extends generally in a proximal direction. In some embodiments, the prosthesis 10 can include one or more proximal anchors 34. The proximal anchors 34 can be positioned along or proximate a proximal end 22 of the frame 20 and can be connected to the frame 20. The proximal anchors 34 can be designed such that when the frame 20 is in an expanded configuration an end or tip 36 of each proximal anchor 34 is positioned radially outward from the frame 20 and extends generally in a distal direction. In some embodiments, one or more anchors 30, 34 can include cushions 38, 39 covering one or more of such anchors.

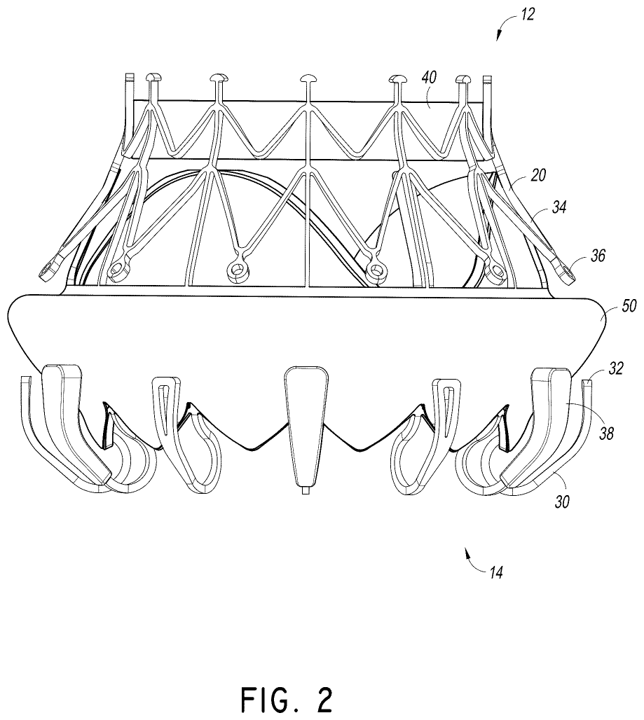

In some embodiments, the cushion 38 can be formed from two separate pieces of material such as an inner portion positioned within a covering such that the covering forms a layer surrounding the inner portion. For example, the inner portion can be wholly contained within the covering. In some embodiments, the inner portion can be formed of a foam material such that the inner portion is at least somewhat compliant and the covering can be formed of a biocompatible, fabric material. The embodiment of FIGS. 1A, 1B and 2 illustrates cushions 38 on alternating distal anchors 30, the cushions 38 extending partially from an end or tip of the anchor 30 towards the connection between the anchor 30 and the frame 20. Use of cushions 38 on alternating distal anchors 30 can maintain a smaller form factor while in the prosthesis 10 is in a contracted state for delivery. As such, for embodiments having twelve distal anchors 30, a total of six distal anchors 30 can have cushions 38 and a total of six distal anchors 30 may not have a cushion 38. The cushions 38 can advantageously increase contact area of the anchors 30 on tissue. This can reduce trauma between the anchor 30 and such tissue. Moreover, this can facilitate growth of tissue in and/or around the anchor 30 in embodiments where the cushions 38 are formed of a material which encourages tissue growth. The cushions 38 on anchors 30 adjacent anchors 30 without a cushion 38 can also beneficially reduce any potential trauma caused by adjacent anchors 30 without a cushion 38.

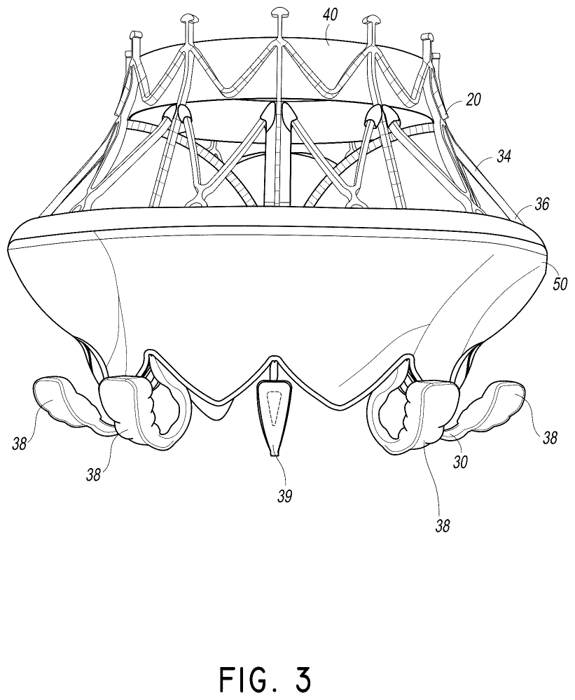

The embodiment of FIG. 3 illustrates cushions 38, 39 on all distal anchors 30. As shown some of the distal anchors 30 include thicker cushions 38 than other distal anchors 30. The cushions 38, 39 can extend the majority or entirety of the length of the anchor 30 from an end or tip of the anchor 30 towards the connection between the anchor 30 and the frame 20. As shown, two distal anchors 30 include thicker cushions 38 with a distal anchor 30 having a thinner cushion 39 positioned therebetween. As such, for embodiments having twelve distal anchors 30, a total of eight distal anchors 30 can have thicker cushions 38 and a total of four distal anchors 30 can include thinner cushions 39. The thicker cushions 38 can be formed of an inner portion and a cover layer, with the inner portion being formed from a compliant material, such as foam, and the covering can be formed of a biocompatible, fabric material. As shown, the inner portion can be positioned only around a portion of the anchor 30 whereas the covering can extend the majority or entirety of the length of the anchor 30. The thinner cushion 39 can be a cover layer with a thinner inner portion or without an inner portion. The inner portion and/or the covering can be formed of a material which encourages tissue growth.

Other configurations of cushions 38, 39 can also be used. For example, in some embodiments, the cushions 38, 39 can be included on proximal anchors 34. In some embodiments, the cushions 38, 39 can be positioned on other portions of the frame 20 such as, but not limited to, one or more of the struts forming the frame 20. The cushions 38, 39 can advantageously increase contact area of the prosthesis 10 on tissue. This can reduce trauma between the frame 20 and such tissue. Moreover, this can facilitate growth of tissue in and/or around the frame 20 in embodiments where the cushions 38, 39 are formed of a material which encourages tissue growth. In some embodiments, the covering of cushions 38, 39 can extend from the annular flap 50 and be formed from materials similar to those of the annular flap 50. The covering of cushions 38, 39 can cover a majority or the entirety of the distal anchors 30 as shown in FIG. 3. In some embodiments, the cushions 38, 39 can be attached to the distal anchors 30 via circumferential stitching about a longitudinal axis of the distal anchor 30.

With reference to the embodiments of FIGS. 1A-3, in some embodiments the prosthesis 10 can include a band 40 along or proximate the proximal end 22 of the frame 20. The band 40 can include features and perform functions similar to those described in U.S. patent application Ser. No. 13/403,929 filed Feb. 23, 2012, titled REPLACEMENT VALVE AND METHOD, published as U.S. Publication No. 2012/0215303, the entire contents of which is incorporated by reference herein.

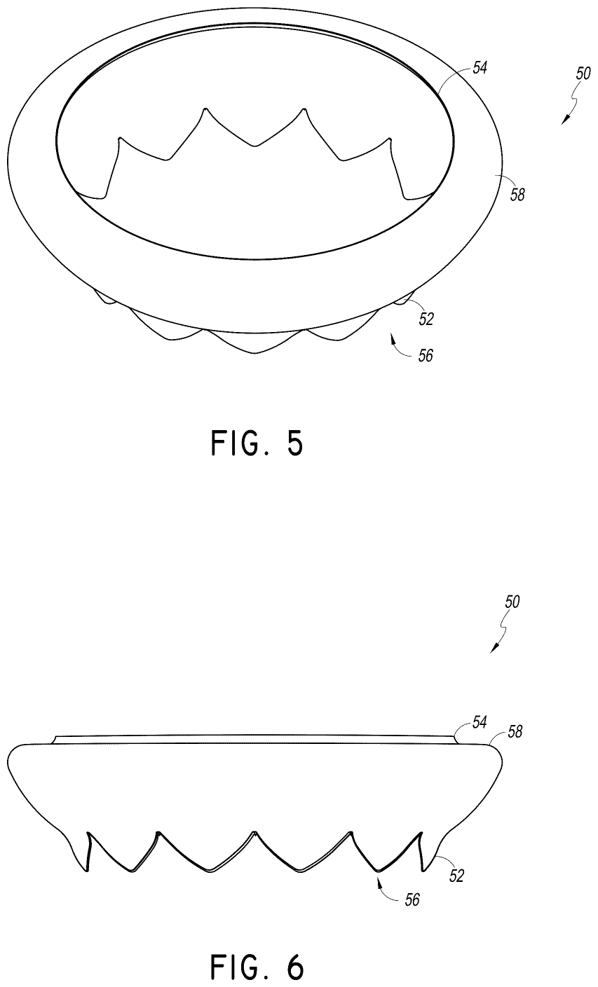

With reference to the embodiments of FIGS. 1A-3, 5 and 6, the prosthesis 10 can include an annular flap 50 which can be positioned around and secured to an exterior of the frame 20. The annular flap 50 can have a distal edge 52 secured at or proximate the distal end 24 of the frame 20 and extend to a proximal edge 54 secured at or proximate an intermediate location, such as the intermediate portion 26, on the frame 20 between the proximal and distal ends 22, 24. In some embodiments, the distal edge 52 of the annular flap 50 can be provided with a shape that generally corresponds to the shape of the frame 20. This can facilitate the securement of the flap 50 to the frame 20. For example, as illustrated in the embodiments of FIGS. 1A-3, 5 and 6, the distal edge 52 can include a generally triangular pattern 56 which follows the generally triangular, zig-zag or undulating pattern of the struts of frame 20 along the distal end 24 of frame 20. Other shapes and/or patterns 56 can be used along the distal edge 52 of the annular flap 50. In some embodiments, the distal edge 52 of the annular flap 50 can have no pattern. In some embodiments the distal edge 52 does not follow the pattern of the struts of the frame 20 and/or can have a different pattern from that of the struts.

In some embodiments, such as the embodiments of FIGS. 1A-3, 5 and 6, the annular flap 50 can have a flange 58. The flange 58 can extend generally radially outward in a direction generally orthogonal to the longitudinal axis 28 extending through the frame 20. In some embodiments, the flange 58 can also project proximally and/or distally. The flange 58 can be used to further prevent or inhibit backflow of fluids around the prosthesis 10. In some embodiments, the flange 58 can be formed from a first layer of resilient material, such as polyethylene terephthalate (PET) or any other biocompatible material, which extends radially outward from the frame 10. In some embodiments, a second layer of resilient material, such as PET or any other biocompatible material, can extend from the first layer in a distal direction towards a distal end 24 of the frame 20. In some embodiments, the first and second layers can be connected together using a suitable mechanism such as adhesives or sutures. In some embodiments, the annular flap 50 can be formed from a single layer of resilient material. In some embodiments, the first and/or second layers can be formed from a deformable material. In some embodiments, the first and/or second layers can be formed from a material which is wholly or substantially fluid impermeable. The annular flap 50 can also include other structures, such as wires formed from resilient materials such as nitinol, to allow at least portions of the annular flap 50 to retain a particular shape. These structures may be positioned on an inner surface of the annular flap 50.

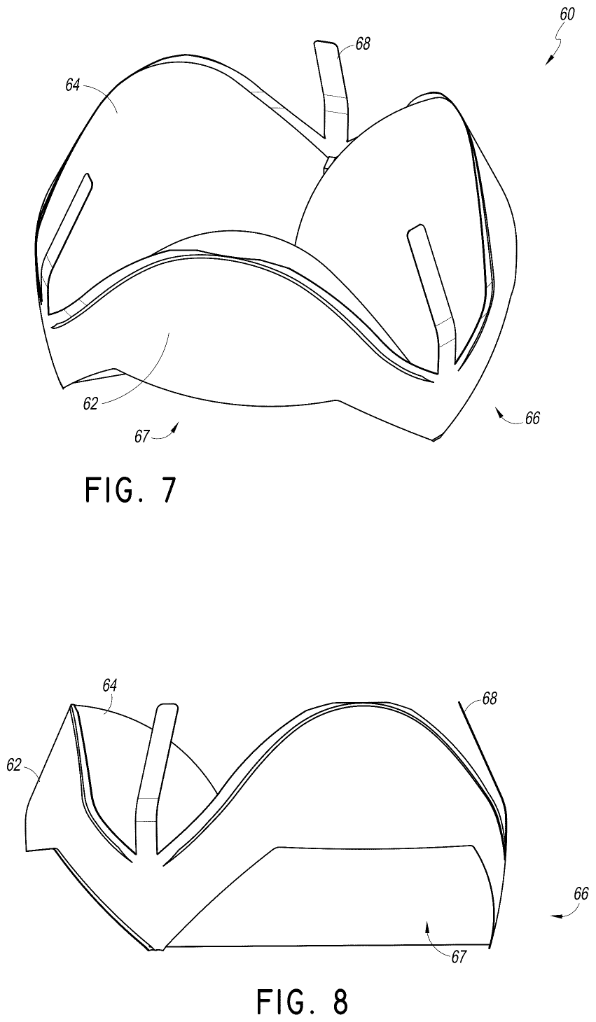

In some embodiments, the flange 58 can be formed when the annular flap 50 is in an expanded configuration. When the flap is in an expanded configuration, such as illustrated in the embodiment of FIG. 6, the radius of the annular flap 50 can decrease distal of the flange 58. As will be described in further detail below, the annular flap 50 can have a first, collapsed or deflated configuration in which the flap 50 is closer to the frame 20 to a second, expanded or inflated configuration in which the flap 50 is spaced further away from the frame 20. The expanded configuration can increase the surface area of the prosthesis 10 and create a barrier to fluid flow exterior to the frame 20 when deployed within a body cavity. The transition from the first configuration to the second configuration, and from the second configuration to the first configuration, can be triggered by blood flow into and out of the interior region of the flap 50, as described further below.

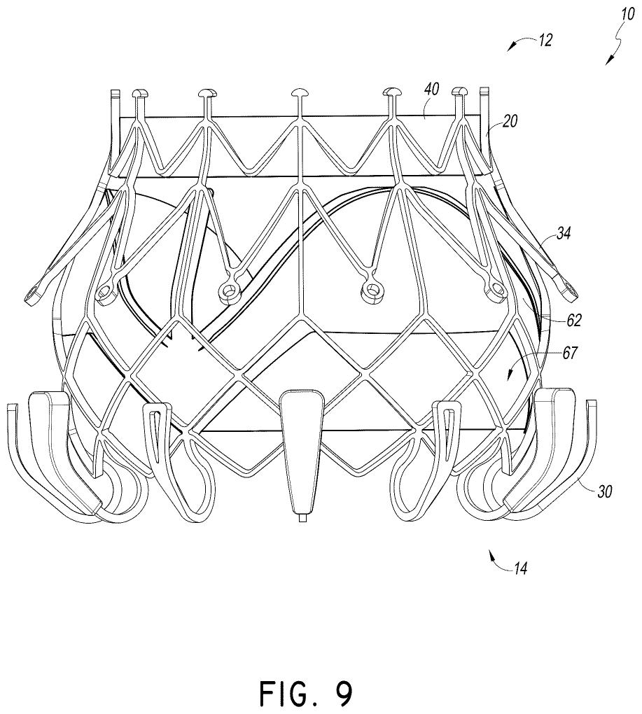

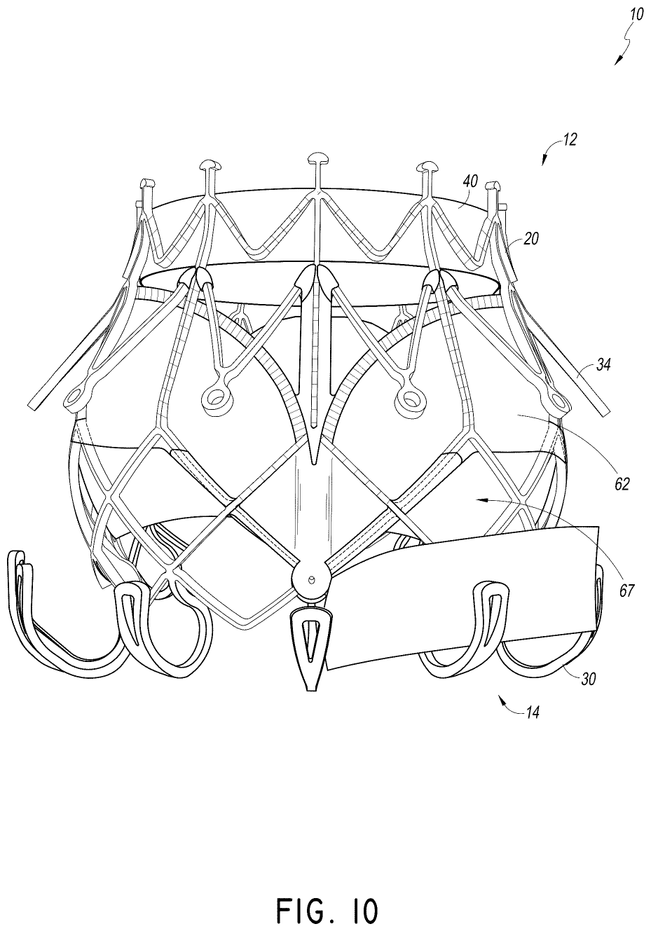

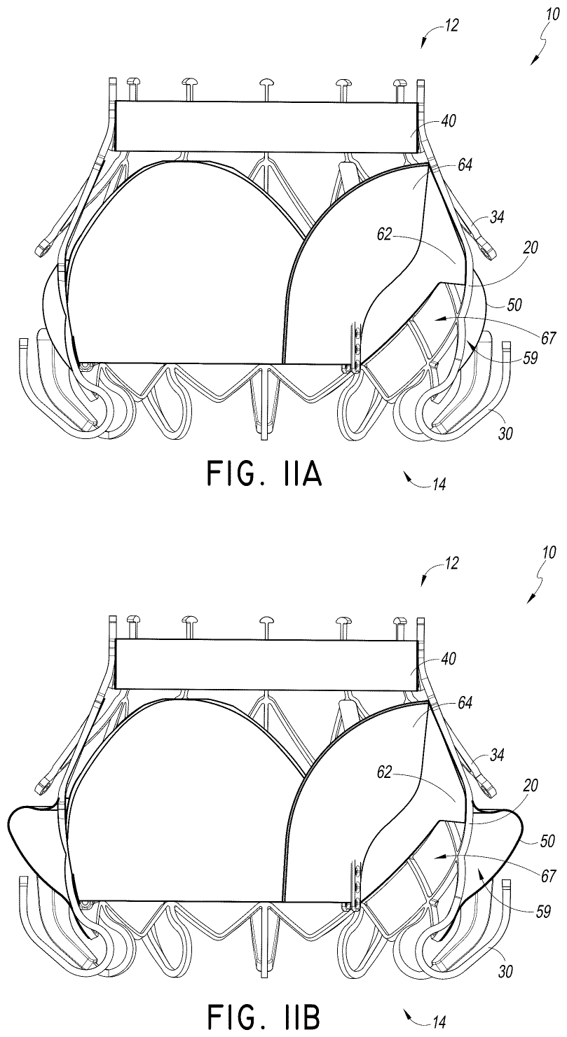

With reference to the embodiments of FIGS. 1A-3 and 7-10, the prosthesis 10 can include a valve body 60 positioned within an interior of the frame 20. In some embodiments, the valve body 60 can include an inner skirt 62 secured to the interior of the frame 20. The valve body 60 can include a plurality of leaflets 64 which can be designed to allow flow in a first direction, such as a proximal to distal direction, while preventing flow in a second direction, such as a distal to proximal direction. In some embodiments, the leaflets 64 have a curved, proximal edge which is fixed to the inner skirt 62 and a distal edge which freely moves. In such embodiments, movement of the distal edges towards and away from each other can allow the valve body 60 to open and close depending on the direction of flow. Accordingly, the valve body 60 can function as a one-way valve such as a mitral valve. In some embodiments, the leaflets 64 are secured to the inner skirt 62. The leaflets 64 and the inner skirt 62 can be manufactured from the same material or from different materials. For example, the inner skirt 62 can be manufactured from a more rigid material than the leaflets 64. In some embodiments, the distal end 66 of the inner skirt 62 can be secured at or proximate the distal end 24 of the frame 20. In some embodiments, such as is illustrated in the embodiments of FIGS. 9 and 10, the distal end 66 of the inner skirt 62 can be positioned slightly proximal of the distal end 24 of the frame 20. This can allow facilitate blood flow around the outside of the inner skirt 62 and into the annular flap 50. The inner skirt 62 can include one or more openings or cutouts 67 positioned along a distal end 66 of the inner skirt 62. This can further facilitate blood flow around the outside of the inner skirt 62. In some embodiments, the valve body 60 can include arms 68 to further secure the valve body 60 to the frame 20.

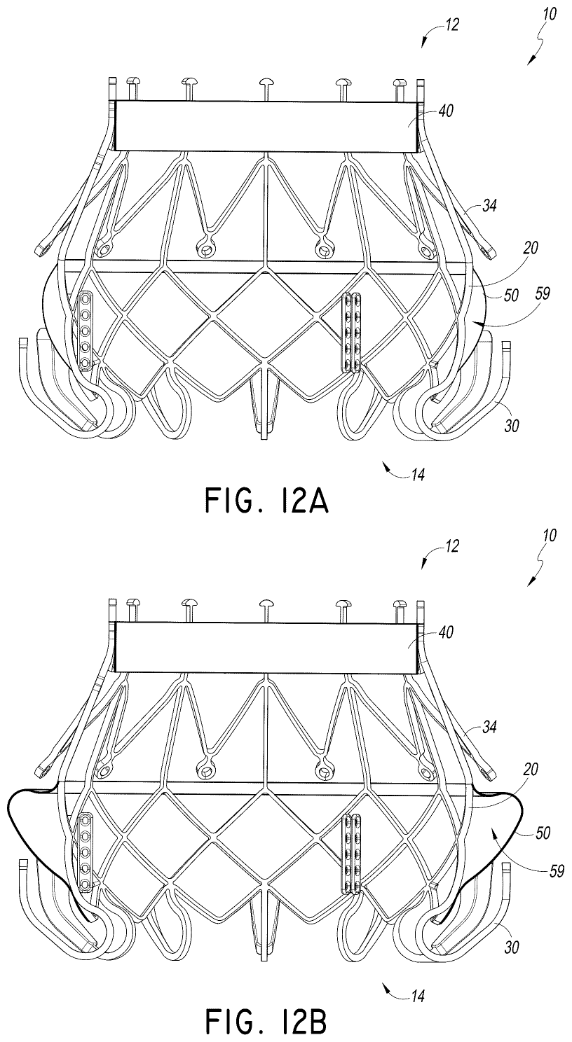

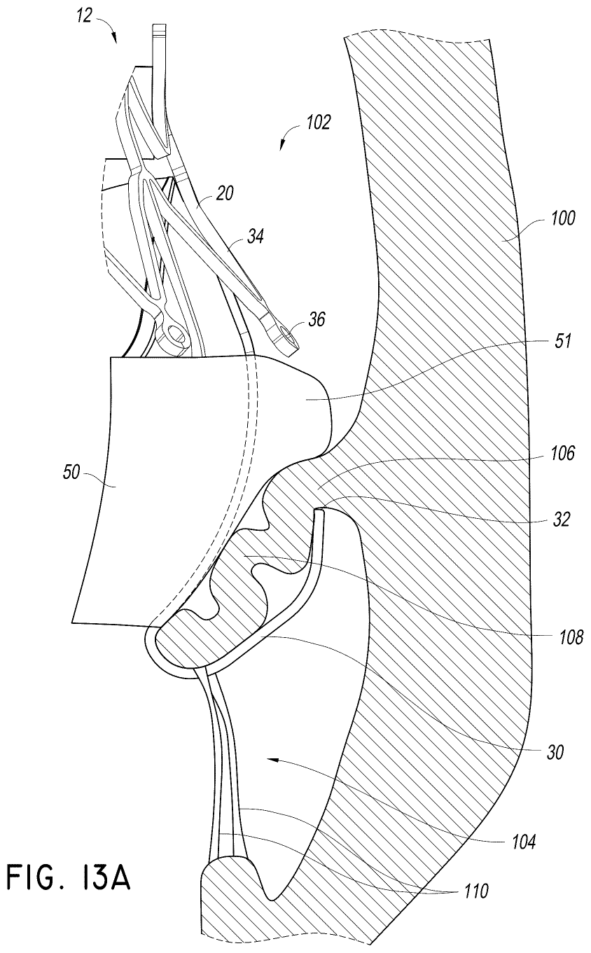

Reference is now made to the embodiments of FIGS. 11A-B and 12A-B which illustrate two configurations of the annular flap. It should be noted that the embodiment of FIGS. 12A-B is similar to the embodiment of FIG. 11A-B with the valve body 60 removed. As shown in the embodiments of FIGS. 11A and 12A, in a first configuration the annular flap 50 is positioned closer to the frame 20. In the event that fluid flows in a second direction, such as a distal to proximal direction, at least a portion of the fluid can enter into an opening between the frame 20 and the annular flap 50, such as opening or cutout 67 formed along the distal end 66 of the inner skirt 62, and collect within a space 59 such that the annular flap 50 takes on the second configuration as shown in the embodiments of FIGS. 11B and 12B. As shown in the embodiments of FIG. 1B, the frame 20 can be positioned within the space 59 between the annular flap 50 and the valve body 60. This effect can be enhanced if the valve body 60 is designed to prevent fluid flow in the second direction (e.g., distal to proximal), such that a substantial portion of fluid is forced around and into the annular flap 50. The annular flap 50 can revert back to the first configuration when fluid flows in a first direction, such as a proximal to distal direction, such that fluid is expelled from within the space 59. In some embodiments, the space 59 can be formed between the inner skirt 62 and the flap 50. For example, both the inner skirt 62 and the flap 50 can be connected to the frame 20 along this region, such as along a proximal edge 54 of the flap 50, such that the inner skirt 62 and flap 50 serve as a barrier to flow of fluid outward from space 59.

Reference is now made to FIG. 13A-15 which illustrate schematic representations of an embodiment of a replacement heart valve 10 positioned within a native mitral valve of a heart 100. A portion of the native mitral valve is shown schematically and represents typical anatomy, including a left atrium 102 positioned above an annulus 106 and a left ventricle 104 positioned below the annulus 106. The left atrium 102 and left ventricle 104 communicate with one another through a mitral annulus 106. Also shown schematically in FIGS. 13A-15 is a native mitral leaflet 108 having chordac tendineae 110 that connect a downstream end of the mitral leaflet 108 to the papillary muscle of the left ventricle 104. The portion of the replacement heart valve 10 disposed upstream of the annulus 106 (toward the left atrium) can be referred to as being positioned supra-annularly. The portion generally within the annulus 106 is referred to as positioned intra-annularly. The portion downstream of the annulus 106 is referred to as being positioned sub-annularly (toward the left ventricle). In the illustrated embodiment, only a part of the foreshortening portion is positioned intra-annularly or sub-annularly, and the rest of the replacement heart valve 10 is supra-annular.

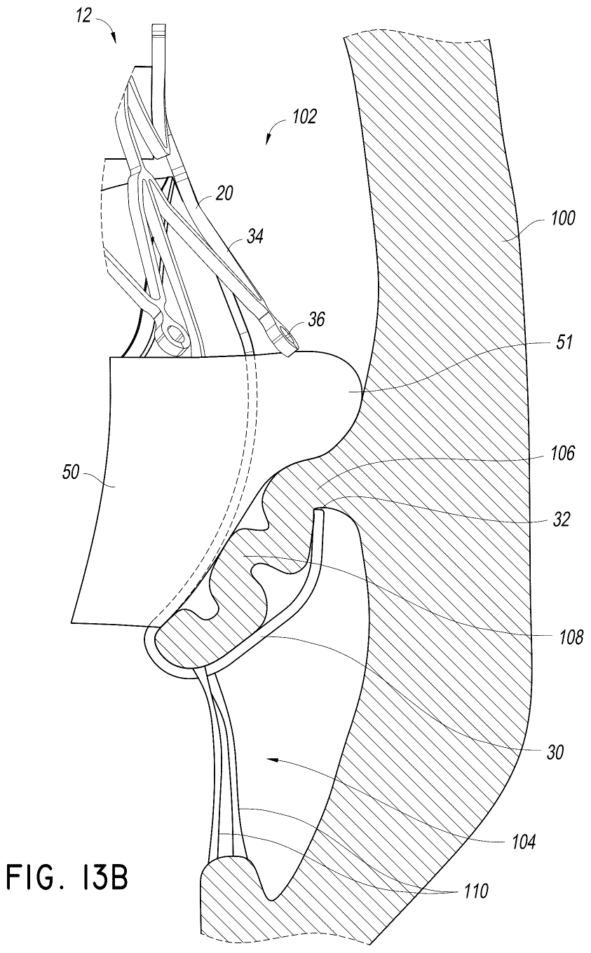

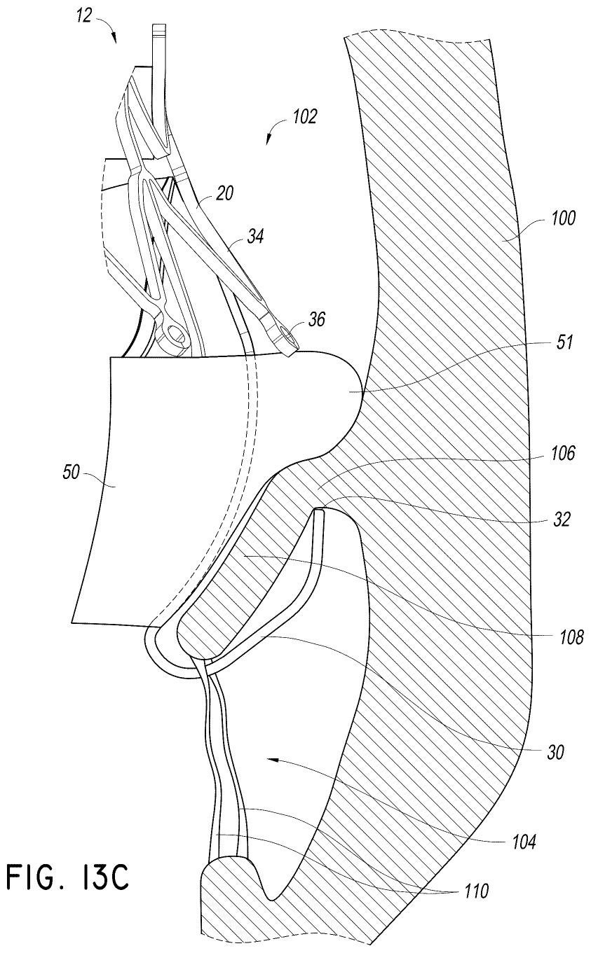

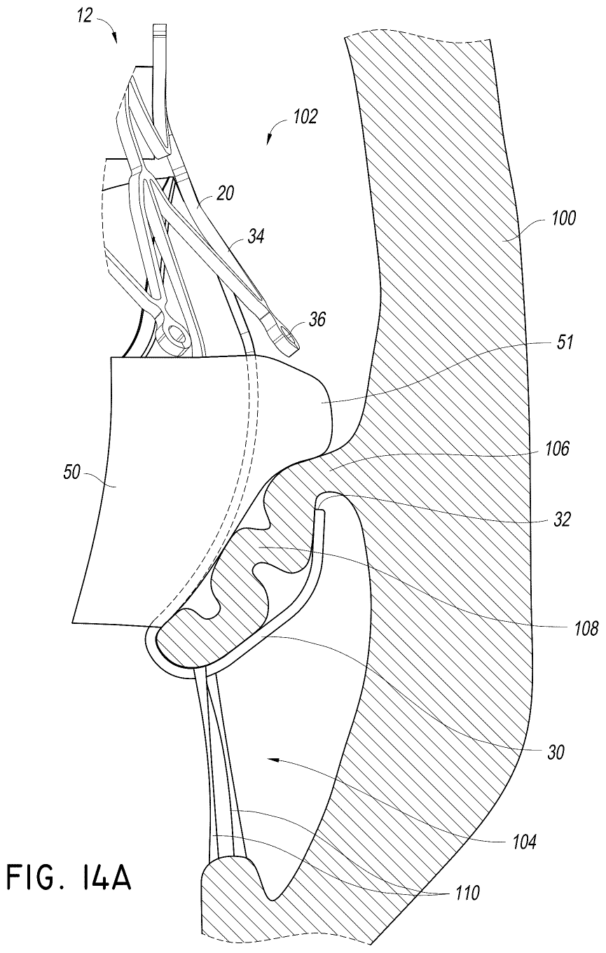

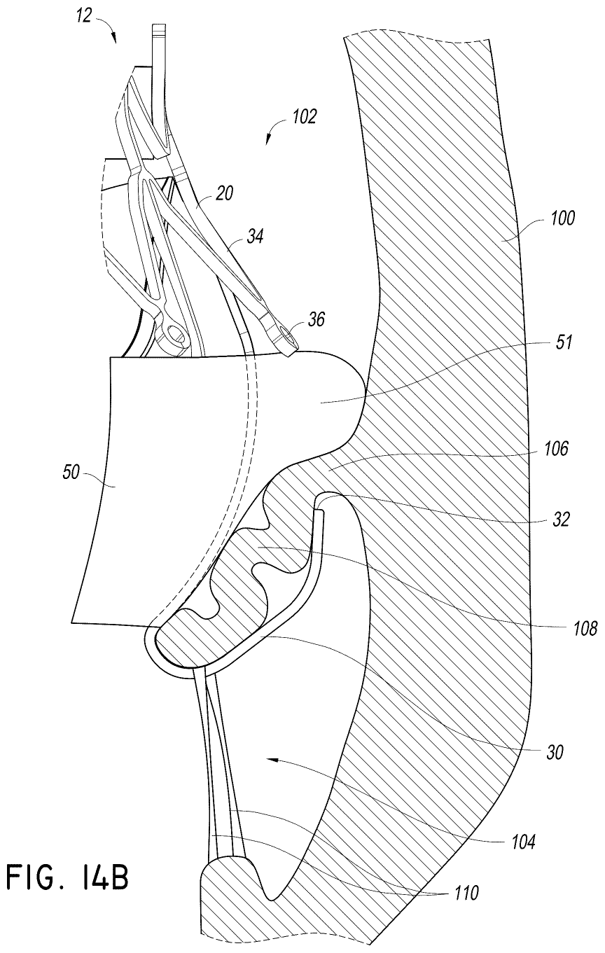

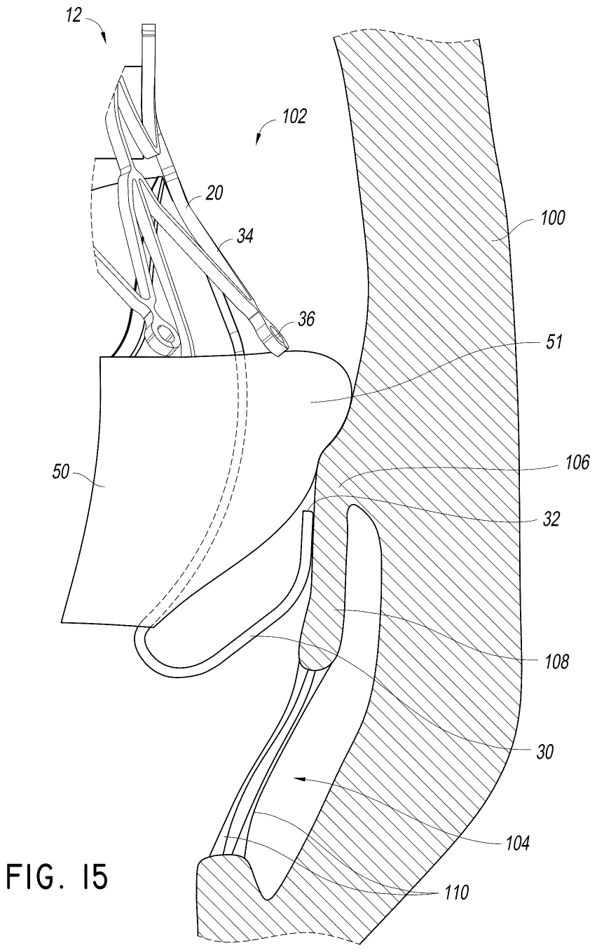

As shown in the situations illustrated in FIGS. 13A-14, the replacement heart valve 10 can be disposed so that the mitral annulus 106 is between the distal anchors 30 and the proximal anchors 34. In some situations, the prosthesis 10 can be positioned such that ends or tips 32 of the distal anchors 30 contact the annulus 106 as shown, for example, in FIGS. 13A-13C. In some situations, the prosthesis 10 can be positioned such that ends or tips 32 of the distal anchors 30 do not contact the annulus 106 as shown, for example, in FIGS. 14A-14B. In some situations, the prosthesis 10 can be positioned such that the distal anchors 30 do not extend around the leaflet 108 as shown in FIG. 15. While FIGS. 13A-15 are described separately below, it should be understood that one or more of the situations illustrated in FIGS. 13A-15 may be present when the prosthesis 10 is positioned at the implantation location, such as a native mitral valve. For example, in some situations the prosthesis 10 may be positioned such that some distal anchors 30 may contact the annulus 106 while other distal anchors 30 may not.

With reference first to the situations illustrated in FIGS. 13A-14B, the replacement heart valve 10 can be positioned so that the ends or tips 32 of the distal anchors 30 are on a ventricular side of the mitral annulus 106 and the ends or tips of 36 the proximal anchors 34 are on an atrial side of the mitral annulus 106. The distal anchors 30 can be positioned such that the ends or tips 32 of the distal anchors 30 are on a ventricular side of the native leaflets beyond a location where chordac tendineae 110 connect to free ends of the native leaflets. The distal anchors 30 may extend between at least some of the chordac tendineae 110 and, in some situations such as those shown in FIGS. 13A-13C, can contact or engage a ventricular side of the annulus 106. It is also contemplated that in some situations, such as those shown in FIGS. 14A and 14B, the distal anchors 30 may not contact the annulus 106, though the distal anchors 30 may still contact the native leaflet 108. In some situations, the distal anchors 30 can contact tissue of the left ventricle 104 beyond the annulus 106 and/or a ventricular side of the leaflets.

During delivery, the distal anchors 30 (along with the frame 20) can be moved toward the ventricular side of the annulus 106 with the distal anchors 30 extending between at least some of the chordac tendineae 110 to provide tension on the chordae tendineae 110. The degree of tension provided on the chordac tendineae 110 can differ. For example, little to no tension may be present in the chordac tendineae 110 as shown in FIG. 13C where the leaflet 108 is shorter than or similar in size to the distal anchors 30. A greater degree of tension may be present in the chordac tendineae 110 as shown in FIGS. 13A and 13B where the leaflet 108 is longer than the distal anchors 30 and, as such, takes on a compacted form and is pulled proximally. An even greater degree of tension may be present in the chordac tendineae 110 as shown in FIGS. 14A and 14B where the leaflets 108 are even longer relative to the distal anchors 30. As shown in FIGS. 14A and 14B, the leaflet 108 is sufficiently long such that the distal anchors 30 do not contact the annulus 106.

The proximal anchors 34 can be positioned such that the ends or tips 36 of the proximal anchors 34 are adjacent the atrial side of the annulus 106 and/or tissue of the left atrium 102 beyond the annulus 106. In some situations, some or all of the proximal anchors 34 may only occasionally contact or engage atrial side of the annulus 106 and/or tissue of the left atrium 102 beyond the annulus 106. For example, as shown in FIGS. 13A and 13B, the proximal anchors 34 may be spaced from the atrial side of the annulus 106 and/or tissue of the left atrium 102 beyond the annulus 106. The proximal anchors 34 could provide axial stability for the prosthesis 10. In some situations such as those shown in FIGS. 13A and 14A, some or all of the proximal anchors 34 may not contact the annular flap 50. This may occur when the annular flap 50 is in a collapsed configuration although it may also occur when the annular flap 50 is in an expanded configuration. In some situations such as those shown in FIGS. 13B, 13C and 14B, some or all of the proximal anchors 34 may contact the annular flap 50. This may occur when the annular flap 50 is in an expanded configuration although it may also occur when the annular flap 50 is in a collapsed configuration. It is also contemplated that some or all of the proximal anchors 34 may contact the atrial side of the annulus 106 and/or tissue of the left atrium 102 beyond the annulus 106

With continued reference to the situations illustrated in FIGS. 13A-14B, the annular flap 50 can be positioned such that a proximal portion 51 of the annular flap 50 is positioned along or adjacent an atrial side of the annulus 106. The proximal portion 51 can be positioned between the atrial side of the annulus 106 and the proximal anchors 34. The proximal portion 51 can extend radially outward such that the annular flap 50 is positioned along or adjacent tissue of the left atrium 102 beyond the annulus 106. The annular flap 50 can create a seal over the atrial side of the annulus 106 when the flap 50 is in the expanded state.

The flap 50 can transition from the collapsed state to the expanded state during systole when pressure in the left ventricle 104 increases. This increased pressure within the left ventricle 104 can cause blood within the left ventricle 104 to be directed to areas of lower pressure, such as the aorta (not shown) and the left atrium 102. As noted above, during systole the valve body 60 may be closed to prevent blood from flowing back into the left atrium 102. A substantial portion of blood can forced around the frame 20 and valve body 60 and into the annular flap 50 such that the flap 50 can expand. Sealing along an atrial side of the annulus 106 can be particularly effective. The left atrium 102 can be at a lower pressure in comparison to the pressure of the space 59 between the annular flap 50 and the valve body 50, which is closer to the pressure of the left ventricle 104. The existence of such a pressure differential between the left atrium 102 and the space 59 during systole can allow the flap 50 to apply a greater force to surrounding tissue within the left atrium 102. During diastole, where blood flows from the left atrium 102 towards the left ventricle 104, the flap 50 can transition from the expanded state back to the collapsed state.

In some situations such as those shown in FIGS. 13A and 14A, the annular flap 50 may not contact the wall of the heart 100. This may occur when the annular flap 50 is in a collapsed configuration although it may also occur when the annular flap 50 is in an expanded configuration. In some situations such as those shown in FIGS. 13B, 13C and 14B, the annular flap 50 may contact the wall of the heart 100. This may occur when the annular flap 50 is in an expanded configuration although it may also occur when the annular flap 50 is in a collapsed configuration. As shown in FIG. 13A-14B, the annular flap 50 can also assist in filling gaps which exist between the leaflet 108 and the frame 20 (portions of which are illustrated in dashed lines).

In some situations such as that shown in FIG. 15, the leaflet 108 may not be captured between the frame 20 (portions of which are shown in dashed lines) and the distal anchors 30. As shown, the anchor 30 may be positioned along an atrial surface of the leaflet 108. The anchor 30 may also be positioned along an inner surface of the annulus 106. It is also contemplated that the anchor 30 may exert a force against the leaflet 108 such that the leaflet 108 is pushed radially outward, relative to the longitudinal axis 28, towards a wall of the heart 100. In such situations, the flap 50 can create a seal intra-annularly and/or along an atrial side of the leaflet 108. In alternative situations (not shown), the flap 50 can create a seal along a ventricular side of the annulus 106. For example, the replacement heart valve 10 may be disposed in the mitral annulus such that a portion of the annular flap 50 is positioned on the ventricular side of the native annulus 106.

As noted above, although the in vivo situations of FIG. 13A-15 have been described separately, it should be understood that one or more of these situations may be present when a prosthesis is positioned at the implantation location, such as a native mitral valve. For example, one or more of the distal anchors 30 may not capture the leaflet 108 whereas the remaining anchors 30 may capture the leaflet 108. As another example, when the prosthesis 10 is positioned within the native mitral valve, the annular flap 50 can contact the wall of the heart 100 along one or more portions of an outermost circumference of the proximal portion 51 and may not contact the wall of the heart 100 along other portions of the outermost circumference of the proximal portion 51. For example, the annular flap 50 may contact the wall of the heart 100 along an approximately 180 degree portion of the outermost circumference of the proximal portion 51 and may not contact the wall of the heart 100 along the remaining, approximately 180 degree portion of the outermost circumference of the proximal portion 51.

Replacement heart valves can be delivered to a patient's heart mitral valve annulus in various ways, such as by open surgery, minimally-invasive surgery, and percutaneous or transcatheter delivery through the patient's vasculature. In some embodiments, the replacement heart valve can be delivered transapically or transfemorally.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

Similarly, this method of disclosure is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A valve prosthesis for implantation at a native heart valve, comprising:
an expandable frame having a first cross-sectional area, the expandable frame configured to radially expand for deployment within a native heart valve annulus;
a valve body positioned within an interior of the expandable frame, the valve body having a plurality of leaflets configured to allow flow in a first direction and inhibit flow in a second direction, the first direction extending from an atrial portion of the valve prosthesis to a ventricular portion of the valve prosthesis and the second direction extending from the ventricular portion to the atrial portion;
a first anchoring feature, the first anchoring feature having a proximal end with a second cross-sectional area and a distal end with a third cross-sectional area, wherein the second cross-sectional area is greater than the first cross-sectional area and the third cross-sectional area, the first anchoring feature configured to facilitate anchoring of the expandable frame within the native heart valve annulus;
a plurality of looped anchors configured to engage native leaflets and to engage the native leaflets between the first anchoring feature and the plurality of looped anchors; and
a second anchoring feature attached to the expandable frame, the second anchoring feature configured to engage a wall of a heart.

2. The valve prosthesis of claim 1, further comprising a skirt surrounding the expandable frame.

3. The valve prosthesis of claim 1, wherein the plurality of looped anchors extend radially outwardly and in an upstream direction.

4. The valve prosthesis of claim 1, wherein the second anchoring feature comprises two struts attached to the expandable frame.

5. The valve prosthesis of claim 4, wherein the two struts connect to form a triangular shape.

6. The valve prosthesis of claim 1, wherein the valve prosthesis is configured to transition from a collapsed configuration to an expanded configuration.

7. The valve prosthesis of claim 1, wherein the first anchoring feature extends radially outward such that the first anchoring feature is positioned along tissue of a native atrium of the heart beyond the native heart valve annulus.

8. A valve prosthesis for implantation at a native heart valve, comprising:
an expandable frame configured to radially expand for deployment within a native heart valve annulus;
a valve body positioned within an interior of the expandable frame, the valve body having a plurality of leaflets configured to allow flow in a first direction and inhibit flow in a second direction, the first direction extending from an atrial portion of the valve prosthesis to a ventricular portion of the valve prosthesis and the second direction extending from the ventricular portion to the atrial portion;
a first anchoring feature configured to facilitate anchoring of the expandable frame within the native heart valve annulus and to exert a force along an at least a portion of an inner wall of a heart;
a plurality of anchors extending from the ventricular portion of the valve prosthesis, the plurality of anchors configured to engage native leaflets and to engage the native leaflets between the first anchoring feature and the plurality of anchors; and
a further anchoring feature attached to the expandable frame, the further anchoring feature configured to engage a wall of the heart.

9. The valve prosthesis of claim 8, wherein when the first anchoring feature exerts a force along the at least a portion of the inner wall of the heart, the at least a portion of the inner wall of the heart exerts a second force on the first anchoring feature.

10. The valve prosthesis of claim 9, wherein when the at least a portion of the inner wall of the heart exerts the second force on the first anchoring feature, the valve prosthesis is inhibited from entirely entering a ventricle of the heart.

11. The valve prosthesis of claim 8, wherein the first anchoring feature extends radially outward such that the first anchoring feature is positioned along tissue of a native atrium of the heart beyond the native heart valve annulus.

12. The valve prosthesis of claim 8, further comprising a skirt surrounding the expandable frame.

13. The valve prosthesis of claim 8, wherein the plurality of anchors extend radially outwardly and in an upstream direction.

14. The valve prosthesis of claim 8, wherein the further anchoring feature comprises two struts attached to the expandable frame.

15. The valve prosthesis of claim 14, wherein the two struts connect to form a triangular shape.

16. The valve prosthesis of claim 15, wherein at least a portion of the further anchoring feature is covered with a fabric.

17. The valve prosthesis of claim 8, wherein the valve prosthesis is configured to transition from a collapsed configuration to an expanded configuration.

18. A valve prosthesis for implantation at a native heart valve, comprising:
an expandable frame comprising a proximal end, a distal end, and a middle region between the proximal end and the distal end, the expandable frame configured to radially expand for deployment within a native heart valve annulus;
a plurality of struts extending upward from the proximal end of the frame, each of the plurality of struts having enlarged tabs;
an anchoring structure positioned along or proximate a proximal end of the expandable frame and connected to the expandable frame, the anchoring structure being configured to facilitate anchoring of the expandable frame within the native heart valve annulus, wherein, when the expandable frame is in an expanded configuration, a contact portion of the anchoring structure is positioned radially outward from the frame;
a plurality of distal anchors attached to the expandable frame and extending from the distal end of the expandable frame, wherein at least a portion of each of the plurality of distal anchors extending generally proximally when the expandable frame is in an expanded configuration, the plurality of distal anchors configured to engage native leaflets and to engage the native leaflets between the anchoring structure and the plurality of distal anchors, wherein at least one of the plurality of distal anchors comprises a flared end to form an enlarged head portion including a covering or cushion;
a valve body positioned within an interior of the expandable frame, the valve body having a plurality of leaflets configured to allow flow in a first direction and prevent flow in a second direction, wherein each leaflet of the plurality of leaflets comprises a proximal edge and a distal edge which feely moves; and
a fabric skirt secured to the expandable frame for providing an improved seal in a region of the native heart valve annulus.

19. The valve prosthesis of claim 18, wherein the expandable frame comprises a first cross-sectional area, at least a portion of the anchoring structure has a second cross-sectional area, and the first cross-sectional area is less than the second cross-sectional area.

20. The valve prosthesis of claim 18, further comprising a second anchoring feature configured to engage a wall of a heart, wherein the second anchoring feature comprises two struts attached to the expandable frame, and wherein the two struts connect to form a triangular shape.
